(19) United States
(12) Patent Application Publication
(10) Pub. No.: US 2004/0170621 A1
(43) Pub. Date: Sep. 2, 2004

(54) Method of resisting osteoclast formation

(76) Inventors: G. David Roodman, Pittsburgh, PA (US); Sun Jin Choi, Wexford, PA (US); Yasuo Oba, Kochi (JP)

Correspondence Address: ECKERT SEAMANS CHERIN & MELLOTT, 600 GRANT STREET, 44TH FLOOR, PITTSBURGH, PA, 15219

(21) Appl. No.: 10/651,674

(22) Filed: Aug. 29, 2003

Related U.S. Application Data
(63) Continuation-in-part of application No. 10/650,277, filed on Aug. 28, 2003.
(60) Provisional application No. 60/407,335, filed on Aug. 30, 2002.

(51) Int. Cl. A61K 48/00; A61K 39/395

(52) U.S. Cl. 424/130.1; 514/44

(57) ABSTRACT

A method of inhibiting osteoclast formation including inhibiting eosinophil chemotactic factor-L expression or activity. This may be accomplished by a number of means, including the use of anti-ECF-L antibody, antisense S-oligonucleotide to ECF-L, mECF-L polyclonal antisera, rabbit preimmune antisera, OPG RANK-Fc and combinations thereof, as well as other inhibiting materials. In another embodiment, osteoclast formation is inhibited by inhibiting RANKL formation. In a further embodiment, a method of inhibiting osteoclast formation is accomplished by means of mECF-L in the presence of RANKL.

### CROSS-REFERENCE TO RELATED APPLICATIONS

This is a Continuation in part of United States Application (Serial Number not yet assigned) filed Aug. 28, 2003 entitled “METHOD OF RESISTING OSTEOCLAST FORMATION” which was a traditional application claiming priority from U.S. provisional application Serial No. 60/407,335 filed Aug. 30, 2002 entitled “METHOD OF RESISTING OSTEOCLAST FORMATION”.

### BACKGROUND OF THE INVENTION

1. Field of the Invention

The present invention relates to a method of inhibiting formation of mature osteoclast cells and, more specifically, relates to blocking eosinophil chemotactic factor-L (ECF-L) and nuclear factor kappa B (RANK) ligand.

2. Description of the Prior Art

Osteoclasts (OCLs) are multinucleated giant cells which resorb bone and are derived from cells in the monocytic lineage. Kurihara N, Chenu C, Miller M, Civin C, Roodman G D, 1990 Identification of committed mononuclear precursors for osteoclast-like cells formed in long term human marrow cultures. Endocrinology 126:2733-2741. A number of factors that control osteoclastogenesis have been reported including soluble cytokines and membrane bound factors on stromal cells and osteoblasts, e.g., the receptor activator of nuclear factor kappa B (RANK) ligand. Roodman G D, 2001 Biology of osteoclast activation in cancer. J Clin Oncology 19:3562-3571. The differentiation of OCLs requires the presence of marrow stromal cells and osteoblasts, and cell-to-cell contact between osteoblast and hematopoietic cells is necessary for inducing differentiation of OCLs. Udagawa N, Takahashi N, Akatsu T, Tanaka H, Sasaki T, Nishihara T, Koga T, Martin T J, Suda T, 1990 Origin of osteoclasts: mature monocytes and macrophages are capable of differentiating into osteoclasts under a suitable microenvironment prepared by bone marrow-derived stromal cells. Proc Natl Acad Sci USA 87:7260-7264. RANK ligand is a critical osteoclastogenic factor that is expressed by osteoblasts and marrow stromal cells in response to several osteotropic factors such as 1,25-dihydroxyvitamin D₃ Biskobing D M, Rubin J, 1993 1,25-Dihydroxyvitamin D3 and phorbol myristate acetate produce divergent phenotypes in a monomyelocytic cell line. Endocrinology 132:862-866., PTH Takahashi N, Yamana H, Yoshiki S, Roodman G D, Mundy G R, Jones S J, Boyde A, Suda T, 1988 Osteoclast-like cell formation and its regulation by osteotropic hormones in mouse bone marrow cultures. Endocrinology 122:1373-1382., and interleukin-11 (IL-11) Elias J A, Tang W, Horowitz M C, 1995 Cytokine and hormonal stimulation of human osteosarcoma interleukin-11 production. Endocrinology 136:489-498. RANK ligand binds to its cognate receptor, RANK, which is found on OCLs and their precursors. However, the genetic events controlling OCL formation from mononuclear precursors have not been fully elucidated. Therefore, to identify genes that regulate OCL differentiation, we developed an OCL precursor cell line (B/T cells) from mice doubly transgenic for the Bcl-X_{L} and Tag genes Hentunen T A, Reddy S V, Boyce B F, Devlin R, Park H R, Chung H, Selander K S, Dallas M, Kurihara N, Galson D L, Goldring S R, Koop B A, Windle J J, Roodman G D, 1998 Immortalization of osteoclast precursors by targeting Bcl-XL and Simian virus 40 large T antigen to the osteoclast lineage in transgenic mice. J Clin Invest 102:88-97., and used it to form homogeneous populations of OCL. Using this precursor cell line and OCL derived from these cells, PCR-selective cDNA subtraction hybridization was performed to identify genes that were up-regulated in OCLs compared with their precursors. Using this differential screening approach, we identified ADAM 8 (a disintegrin and metalloproteinase) as an OCL stimulatory factor, which can increase mouse OCL formation and bone resorption. Choi S J, Han J H, Roodman G D, 2001 ADAM 8: A novel osteoclastic stimulating factor. J Bone Miner Res 16:814-822. We now report the identification and characterization of a novel osteoclastogenic cytokine, eosinophil chemotactic factor-L (ECF-L), which is overexpressed in OCLs. ECF-L was originally identified as a chemoattractant factor produced by mouse splenocytes that enhances chemotaxis of eosinophils, and attracts not only eosinophils but also T-lymphocytes and bone marrow cells. Owhashi M, Arita H, Hayai N, 2000 Identification of a novel eosinophil chemotactic cytokine (ECF-L) as a chitinase family protein. J Biol Chem 275:1279-1286. ECF-L is expressed in spleen, bone marrow, lung, and heart. However, the role of ECF-L in osteoclastogenesis was previously unknown. Chemokines have been characterized on the basis of their chemotactic activity on leukocytic cells with little attention focused on their capacity to affect other cellular functions. Chemokines function as key mediators promoting the recruitment, proliferation, and activation of vascular and immune cells. In general, the □ chemokine subfamily members, including IL-8, chemoattract and activate neutrophils and T cells, but not monocytes Owhashi M, Arita H, Hayai N, 2000 Identification of a novel eosinophil chemotactic cytokine (ECF-L) as a chitinase family protein. J Biol Chem 275:1279-1286., whereas many of the □ chemokine group, such as macrophage inflammatory protein-1□(MIP-1□, MIP-1□, RANTES, and MCP-1 acts as chemoattractants and activators of monocytes, but not neutrophils. Schall T J, Bacon K, Toy K J, Goeddel D V, 1990 Selective attraction of monocytes and T lymphocytes of the memory phenotype by the cytokine RANTES. Nature 347:669-671; Matsushima K, Larsen C G, DuBois G C, Oppenheim J J, 1989 Purification and characterization of a novel monocyte chemotactic and activating factor produced by a human myelomonocytic cell line. J Exp Med 169:1485-1490; Rollins B J, Walz A, Baggiolini M 1991 Recombinant human MCP-1/JE induces chemotaxis, calcium flux, and the respiratory burst in human monocytes. Blood 78:1112-1116. MIP-1□, MIP-1□, and RANTES also exhibit chemoattractant potential for T lymphocytes Taub D D, Conlon K, Lloyd A R, Oppenheim J J, Kelvin D J. 1993, Preferential migration of activated CD4+ and CD8+ T cells in response to MIP-1 alpha and MIP-1 beta. Science 260:355-358., whereas RANTES, and to lesser extent MIP-1□, can act as a chemoattract for eosinophils. Rot A, Krieger M, Brunner T; Bischoff S C, Schall T J, Dahinden C A, 1992 RANTES and macrophage inflammatory protein 1 alpha induce the migration and activation of normal human eosinophil granulocytes. J Exp Med 176:1489-1495. Recently, we reported that the chemokine MIP-1□ is a potent osteoclastogenic factor that acts directly on OCL precursors and enhances OCL formation and bone resorption. Choi S J, Cruz J C, Craig F, Chung H, Devlin R D, Roodman G D, Alsina M, 2000 Macrophage inflammatory protein-1□ is a potential osteoclast stimulatory factor in myeloma. Blood 996:671-675; Han J H, Choi S J, Kurihara N, Koide M, Oba Y Roodman G D, 2001 Macrophage inflammatory protein-1□ is an osteoclastogenic factor in myeloma that is independent of receptor activator of nuclear factor kB ligand. Blood 97:3349-3353. In this study, we report the effects of ECF-L on OCL formation and/or activation which were previously unknown.

It has been suggested previously by the present inventors that ECF-L is a potent mediator of OCL formation which acts in the later stages of OCL differentiation. Oba-Choi-Roodman., abstract (presentation number M287) American Society of Bone and Mineral Research 2001 Meeting (delivered electronically Aug. 14, 2001).

It has also been suggested previously by one of the present inventors that bone is a frequent site of cancer metastasis which can result in bone destruction or no bone formation. Bone destruction, mediated by factors produced or induced by tumor cells, stimulate formation and activation of osteoclasts (OCLs), which are the normal bone-resorbing cells. Several factors, including interleukin (IL)-1, IL-6, receptor activator of the NF-kappaB (RANK) ligand, parathyroid hormone-related protein (PTHrP) and macrophage inflammatory protein-1-alpha (MIP-1∀) are all said to be implicated as factors to enhance osteoclast formation and bone destruction in patients with newplasia. Roodman, Biology of Osteoclast Activation in Cancer, Journal of Clinical Oncology, Vol. 19, No. 15, pp. 3562-3571, (Aug. 1, 2001).

In spite of the foregoing teachings, there remains a very real and substantial need for a method of minimizing the negative effects of osteoclast formation by inhibiting the formation of mature osteoclast cells.

### SUMMARY OF THE INVENTION

A method of inhibiting osteoclast formation including inhibiting eosinophil chemotactic factor-L activity. This may be accomplished by a number of means, including the use of ECF-L antibody, antisense S-oligonucleotide to ECF-L, mECF-L polyclonal antisera, rabbit preimmune antisera, OPG RANK-Fc and combinations thereof, as well as other inhibiting materials. In another embodiment, osteoclast formation is inhibited by inhibiting RANKL formation. In a further embodiment, a method of inhibiting osteoclast formation is accomplished by means of inhibiting mECF-L activity in the presence of RANKL. In preferred embodiments, the anti-ECF-L antibody, or active fragment thereof, is a monoclonal antibody, including but not limited to, human and humanized antibodies. In further preferred embodiments, such antibodies and fragments inhibit or neutralize ECF-L activity and thereby inhibit osteoclast formation. Such antibody fragments include, but are not limited to, scFV, Fab and F(ab′)2 fragments.

It is an object of the present invention to provide a method for resisting formation of osteoclast cells.

It is an object of the present invention to provide such a method which inhibits the function of mature osteoclast cells.

It is a further object of the present invention to provide such a method which inhibits the normal functioning of eosinophil chemotactic factor-L (ECF-L) and the influence of RANK ligand.

It will be appreciated that the present invention provides an effective means of therapeutic use in vivo in humans exhibiting OCL formation based upon ECF-L or RANKL

It is an object of the present invention to provide a therapeutic method for inhibiting the generation of OCLs based upon ECL-F or RANKL.

These and other objects of the invention will be more fully understood from the following description of the invention with reference to the drawings appended hereto.

### BRIEF DESCRIPTION OF THE DRAWINGS

FIGS. 1 and A, respectively, illustrate a plot of TRAP(+) MNCc (mouse osteoclast) versus ECF-L concentrations for two conditions with data for the empty vector (EV) and ECF-L.

FIGS. 2A and B illustrate, respectively, varying concentration for empty vector and ECF-L for each condition plotted against 23c6(+) cells (human osteoclasts) formed.

FIGS. 3A and B illustrate, respectively, factors related to bone resorption as reflected by pit numbers for both EV and ECF-L in terms, respectively, of number of pits and area of pits.

FIG. 4 illustrates a plot for EV and ECF-L if the percentage increase of TRAP(+) MNCs formed as related to time.

FIG. 5 illustrates a plot of sense and antisense oligonucleotide to ECF-L versus TRAP(+) MNCs formation.

FIGS. 6A and B, respectively, illustrate in situ hybridization of ECF-L mRNA performed using antisense and sense probes for ECF-L mRNA with mononuclear cells and MNCs that had less than five nuclei expressed ECF-L mRNA indicated by arrows and MNC that had more than 10 nuclei that did not express ECF-L indicated by arrow heads.

FIG. 7 illustrates a western blot analysis of ECF-L expression in murine bone marrow cultures and 293 cells transiently transfected with the mECF-L cDNA.

FIGS. 8A and B show plots of control and ECF-L antisera, respectively, for 1,25-(OH)₂D₃ and RANKL stimulated osteoclast formation.

FIGS. 9A and B illustrate plots of osteoclast formation induced by control Fc and ECF-L Fc for, respectively, in the presence of 10⁻¹⁰ M 1,25-(OH₂D₃) or 2.5 ng/ml RANKL.

FIG. 9C shows the effect of ECF-L on RANKL mRNA expression with GAPDH being employed as an internal control for RT-PCR.

FIG. 9D illustrates the effects of ECF-L condition media on RANKL protein expression and the ratio of the RANKL band to the ∃-actin band.

FIG. 10 is a plot of the effect of media, ECF-L and ECF-L combined with ECF-L antisera on the chemotaxes of osteoclast precursors.

### DESCRIPTION OF THE PREFERRED EMBODIMENTS

To investigate the molecular mechanisms that control osteoclastogenesis, we developed an immortalized osteoclast (OCL) precursor cell line that forms mature OCLs in the absence of stromal cells and used PCR select cDNA subtraction to identify genes that are highly expressed in mature OCLs compared to OCL precursors. Eosinophil Chemotactic Factor-L (ECF-L), a previously described chemotactic factor for eosinophils, was one of the genes identified. Conditioned media from 293 cells transfected with mECF-L cDNA increased OCL formation in a dose-dependent manner in mouse bone marrow cultures treated with 10⁻¹⁰M 1,25-(OH)₂D₃. OCLs derived from marrow cultures treated with ECF-L conditioned media formed increased pit numbers and resorption area per dentin slice compared to OCLs induced by 1,25-(OH)₂D₃ (p<0.01). Addition of an antisense S-oligonucleotide to mECF-L inhibited OCL formation in murine bone marrow cultures treated only with 10⁻¹⁰M 1,25-(OH)₂D₃ compared to the sense S-oligonucleotide control. Time course studies demonstrated that ECF-L acted at the later stages of OCL formation, and chemotactic assays showed that mECF-L increased migration of OCL precursors. mECF-L mRNA was detectable in mononuclear and multinucleated cells by in situ hybridization. Recombinant mECF-L stimulated murine OCL formation in the presence of 1,25-(OH)₂D₃. Interestingly, a neutralizing antibody to ECF-L blocked RANKL or 10⁻¹⁰M 1,25-(OH)₂D₃-induced OCL formation in mouse bone marrow cultures. Taken together, these data demonstrate ECF-L is a previously unknown factor that is a potent mediator of OCL formation, and is an important factor acting at the later stages of OCL formation.

### Materials and Methods

Materials

In order to verify the effectiveness of the present invention, a series of experiments were performed.

Receptor activator of nuclear factor kB ligand (RANKL) (Immunex, Seattle, Wash., USA) and 1,25-dihydroxyvitamin D₃ [(1,25-(OH)₂D₃), Teijin (Tokyo, Japan)] were generously provided for these experiments. Restriction enzymes, Taq polymerase, fetal calf serum (FCS), and tissue culture media were purchased from Life Technologies (Grand Island, N.Y., USA). All other chemicals were obtained from Sigma (St. Louis, Mo., USA). Chemotactic assay plates were purchased from Corning Costar (Cambridge, Mass., USA).

PCR-selective Subtraction Screening of OCL Precursors and Mature OCLs

OCL precursors and OCLs were prepared from B/T cells as previously described in detail. Hentunen T A, Jackson S H, Chung H, Reddy S V, Lorenzo J, Choi S J, Roodman G D, 1999 Characterization of immortalized osteoclast precursors developed from mice transgenic for both Bcl-XL and Simian virus 40 large T antigen. Endocrinology 140:2954-2961.PCR-selective cDNA subtraction screening for genes that were differentially overexpressed in mature OCLs rather than OCL precursors was performed as previously described Choi S J, Han J H, Roodman G D, 2001 ADAM 8: A novel osteoclastic stimulating factor. J Bone Miner Res 16:814-822 using a PCR-based subtraction kit (#K1804-1) (Clontech, Palo Alto, Calif., USA). Eighteen bands, which were overexpressed in mature OCLs, were reamplified by secondary PCR. After subcloning of the PCR products into the TA vector (Invitrogen, Carlsbad, Calif., USA), the DNA sequences were determined and compared with the DNA sequence database in the National Center for Biotechnical Information (NCBI) to identify them.

Construction of a Full-length Murine ECF-L cDNA

The full-length ECF-L cDNA was generated by PCR using the mouse OCL cDNA as a template and specific primers sets for mECF-L (5′-ACACCATGGCCAAGCTCATT-3′ (sense) and 5′-TGCAGAATGCGCTGTGGAAA-3′ (antisense)). The PCR conditions were 94° C. for 30 sec, 60° C. for 30 sec, 72° C. for 2 min and 40 cycles. The PCR product was subcloned into the TA vector and sequenced. The cDNA was digested with EcoRI and cloned into the mammalian expression vector pcDNA3 (Invitrogen) and transfected into 293 cells to express mECF-L.

Murine OCL-like Multinucleated Cell Formation and Bone Resorption Assays

Mouse bone marrow cultures were performed as previously described to assess the effect of ECF-L on OCL formation/activation Choi S J, Reddy S V, Devlin R D, Menaa C, Chung H, Boyce B F, Roodman G D, 1999 Identification of human asparaginyl endopeptidase (legumain) as autocrine inhibitor of osteoclast formation and bone resorption. J Biol Chem 274:27747-27753. Briefly, freshly isolated mouse bone marrow cells (10⁶/ml in □-Minimal Essential Media (MEM) containing 10% FCS/well in 48 well plates) were cultured for 6-9 days. At the end of culture period, the cells were fixed and stained for tartrate resistance acid phosphatase (TRAP), using a TRAP staining kit (#A-367) (Sigma) to identify OCL-like multinucleated cells (MNCs). MNCs were counted with an inverted microscope. In selected experiments, conditioned media from 293 cells transfected with the mECF-L cDNA were added to marrow cultures for the first 2 days, days 2-4, days 4-6, or for the entire culture period. The cultures were continued for a total of 7 days, and the number of TRAP(+) MNCs formed was determined. For pit formation assays, murine bone marrow cells were cultured on sperm whale dentin slices in 48-well plates. After 8 days of culture, the dentin slices were fixed and stained for TRAP, and the number of TRAP(+) MNCs was scored. The cells on the dentin slices were removed gently by rubbing the slices between the thumb and first finger, and the number of bone resorption pits and area resorbed were measured by image analysis techniques as previously described Yates A J, Oreffo R O, Mayer K, Mundy G R, 1991 Inhibition of bone resorption by inorganic phosphate is mediated by both reduced osteoclast formation and decreased activity of mature osteoclasts. J Bone Miner Res 6:473-478.

Human OCL Formation Assays

Nonadherent human bone marrow mononuclear cells were obtained from normal donors as described previously Han J H, Choi S J, Kurihara N, Koide M, Oba Y, Roodman G D, 2001 Macrophage inflammatory protein-1□ is an osteoclastogenic factor in myeloma that is independent of receptor activator of nuclear factor kB ligand. Blood 97:3349-3353 and tested for their capacity to form OCL-like multinucleated cells (MNCs) in long-term marrow cultures. These studies were approved by the Institutional Review Board of the University of Pittsburgh. The human ECF-L EST clone (AI93402) was identified by a homology search with mouse ECF-L cDNA and purchased from ATCC. DNA sequence analysis was performed to confirm the identity of the hECF-L cDNA, and the insert cDNA was subcloned into the pcDNA3 mammalian expression vector. Conditioned media from 293 cells transfected with the hECF-L cDNA were added to marrow cultures weekly. At the end of the 3 weeks culture period, the number of MNCs that crossreacted with the 23c6 monoclonal antibody was determined. The 23c6 monoclonal antibody identifies OCL-like cells that express calcitonin receptors and resorb bone Schall T J, Bacon K, Toy K J, Goeddel D V, 1990 Selective attraction of monocytes and T lymphocytes of the memory phenotype by the cytokine RANTES. Nature 347:669-671.

Effects of Antisense (AS) and Sense (SS) Oligonucleotides to mECF-L on OCL Formation

To determine if native ECF-L was involved in OCL formation, we designed AS and SS S-oligonucleotides (5′-AAGAATGAGCTTGGCCATGGTGTCTTCACG-3′ and 5′-CGTGAAGACACCATGGCCAAGCTCATTCTT-3′) that included the ATG and ribosome binding site of the mECF-L gene. The AS and SS oligonucleotides were added at varying concentrations to mouse bone marrow cultures stimulated with 10⁻⁹ M 1,25-(OH)₂D₃. Every 2 days, half of the media was replaced with the fresh media containing the SS-oligonucleotide and 10⁻⁹ M 1,25-(OH)₂D₃. At the end of the culture periods, the cells were fixed and stained for TRAP activity, and the number of TRAP(+) MNCs was determined.

In situ Hybridization

In situ hybridization was performed according to the method of Nomura et al. Nomura S, Hirakawa K, Nagoshi J, Hirota S, Kim H M, Takemura T, Nakase T, Takaoka K, Matsumoto S, Nakajima Y, Takebashi K, Takano-Yamamoto T, Ikeda T, Kitamura Y, 1993 Method for detecting the expression of bone matrix protein by in situ hybridization using decalcified mineralized tissue. Acta Histochem Cytochem 26:303-309. Digoxigenin (DIG)-labeled single-strand antisense and sense cRNA probes to mouse ECF-L were prepared using a DIG RNA labeling kit (Roche Diagnostics, Mannheim, Germany). Freshly isolated mouse bone marrow cells were cultured in Lab-Tek 4-chamber slides (Nalge Nunc International, Naperville, Ill., USA) in the presence of 10⁻⁹ M of 1,25-(OH)2D3. After 9 days of culture, the cells were fixed with 4% paraformaldehyde, rehydrated and incubated with 2 □g/ml of proteinase K for 2 min at 37° C. The cells were then treated with 0.2 M HCl for 10 min at room temperature to minimize the non-specific signals through quenching the intrinsic alkaline phosphatase activities. The slides were dehydrated with ethanol, then hybridized with hybridization solution containing either a sense or antisense cRNA probe. The slides were washed with 2×SSC and then 0.2×SSC for 15 min at 50° C. The hybridization signals were detected with a DIG nucleic acid detection kit (Roche Diagnostics, Mannheim, Germany). The sense cRNA probe was hybridized as a control. The slides were counterstained with 0.5% methyl green and imaged.

Expression and Purification of mECF-L in E.coli

Recombinant mECF-L (rmECF-L) was expressed in the BL21 E. coli strain using the pET14b expression vector system (Novagen, Inc., Madison, Wis.) according to the manufacturer's protocol. The nucleotide sequence encoding the mECF-L cDNA was amplified by PCR with sense primers (5′-CGAGGATCCGATGGCCAAGCTCATTCTTGTC-3′) and antisense primers (5′-CGAGGATCCTCAATAAGGGCCCTTGCAACT-3′) (underlined sequences represent BamHI site.) The PCR product was digested with BamHI site and then cloned into the pET 14b vector in frame with the 6×His tag. The plasmid construct was transformed into the BL21 (DE3) E.coli, and the recombinant ECF-L was induced by treatment with 0.5 mM IPTG for 4 hours. The cells were pelleted by centrifugation, washed with PBS and resuspended in His buffer containing 8 M urea. After sonication and centrifugation, the supernatant was loaded onto Ni-NTA Superflow bulk resin (Qiagen, Valencia, Calif., USA) and the 6×His-r ECF-L fusion protein was eluted with a 50-100 mM imidazole gradient. The eluent was dialyzed against milli Q water and injected into rabbit to generate the anti ECF-L polyclonal sera.

Western Blot Analysis for mECF-L in Conditioned Media from Mouse Bone Marrow Cultures

Polyclonal antisera against rmECF-L were raised in rabbits as previously described Choi S J, Devlin R D, Menaa C, Chung H, Roodman G D, Reddy S V, 1998 Cloning and identification of human Sca as a novel inhibitor of osteoclast formation and bone resorption. J Clin Invest 102:1360-1368.and used to determine mECF-L expression in murine bone marrow culture treated with 1,25(OH)₂D₃ by immunoblot analysis. Conditioned media from mouse bone marrow cultures or 293 cells transiently transfected with mECF-L cDNA were harvested and concentrated 30-fold using a Microcon YM-10 (Millipore Corp, Bedford, MAss., USA) filter. Samples were subjected to SDS-PAGE, and then transferred to nitrocellulose membranes. After blocking, the membranes were incubated with polyclonal antibody to mECF-L at 1:2500 dilution for 1 h. Horseradish peroxidase-conjugated goat anti-rabbit IgG (Sigma) was used as a secondary antibody (1:10,000), and the blots were developed with an enhanced chemoluminescent (ECL) system (Pierce, Rockford, Ill., USA) in Kodak X-ray films.

Neutralizing Effects of mECF-L Antisera on OCL Formation in Mouse Bone Marrow Cultures

To confirm the role of endogenous ECF-L on OCL formation/activation, we tested the effects of mECF-L antisera on OCL formation. The anti mECF-L polyclonal antisera or rabbit preimmune antisera (1:1,000-1:10,000) were added to mouse bone marrow culture treated with 10⁻⁹ M 1,25-(OH)₂D₃ or 20 ng/ml RANKL. Every 2 days, half of the media was replaced with fresh media containing the antisera. At the end of the culture periods, the cells were fixed and stained for TRAP activity, and the number of TRAP(+) MNCs was determined.

Production and Purification of Recombinant ECF-L-Fc Fusion Protein

ECF-L cDNA was generated by PCR using T7 and antisense primer (5′-ATCGTAATCCATAAGGGCCCTTGCAACTTG-3′), and the EcoRV-digested PCR-product was fused with the Fc coding domain of human IgG1. The mECF-L-Fc construct was stably transfected into 293 cells using a CaPO₄ mammalian transfection kit (Stratagene, La Jolla, Calif., USA) according to the manufacturer's protocol. One hundred microgram of mECF-L-Fc fusion protein was purified from 1 L of 293 cells conditioned media by protein G affinity chromatography (Roche Diagnostics). The effects of purified mECF-L-Fc fusion protein on OCL formation/activation was tested in murine bone marrow cultures as described above.

RT-PCR and Western Blot Analysis of RANKL Expression in Mouse Bone Marrow Cultures Treated with mECF-L

Mouse bone marrow cells (1.2×10⁷/well) were cultured with mECF-L conditioned media in 6-well plates for 2 days. Total RNA was extracted with RNA-BEE (Tel Test, Friendswood, Tex.) according to the manufacturer's protocol, and the expression levels of mouse RANKL mRNA were determined by RT-PCR analysis. The PCR conditions were 94° C. for 30 sec, 58° C. for 30 sec, 72° C. for 1 min and 28 cycles. PCR primer sequences for mouse RANKL are as follows: sense primers, 5′-GAAGGTACTCGTAGCTAAGG-3′ (sense) and 5′-GGCTATGTCAGCTCCTAAAG-3′(antisense). GAPDH was used as an internal control using primer sequences 5′-ACCACAGTCCATGCCATCAC-3′ (sense) and 5′-TCCACCACCCTGTTGCTGTA-3′ (antisense).

Western blot analysis was used to determine the effects of ECF-L conditioned media on RANKL expression in mouse bone marrow cultures. Mouse bone marrow cells (10⁶ cells) were cultured with 10⁻¹⁰M 1,25-(OH)₂D₃ for 2 days in the presence and absence of 10% mECF-L conditioned media and mECF-L antibody at 1:1,000 dilution. At the end of the culture period, mouse bone marrow cells were lysed with 200 μl of sodium dodecyl sulfate (SDS) lysis buffer and subjected to Western blot analysis as described above. Anti-RANKL polyclonal antibody (R&D Systems, Minneapolis, Minn.) was used as a primary antibody at 1:10,000 dilution. After 1 hour incubation, HRP conjugated antigoat IgG (Santa Cruz Biotechnology, Inc, Santa Cruz, Calif.) was hybridized as a secondary antibody and visualized with ECL on X-ray film. The blot was stripped with a buffer containing SDS and β-mercaptoethanol and reprobed with antibody to β-actin (Santa Cruz Biotechnology) as a control for equal loading. The bands were quantified with a densitometer, and the ratio of RANKL to β-actin was calculated.

Chemotaxis Assays

Chemotaxis assays were performed using 24-well Transwell chambers (8 □m) (Coming Costar, Cambridge, MAss.). Mouse bone marrow cells (2×10⁶/200 □l) were plated in the upper well and 400 □l of 10% FBS-□MEM containing 400 ng/ml of recombinant ECF-L-Fc protein were added to the lower well. ECF-L antisera were added to the media at 1:1000 dilution for control cultures. The cells were incubated for 3-5 hrs, and then the upper wells were removed. To identify OCL precursors that migrated into the lower well, 25 ng/ml of hRANKL were added in the lower well. The cells were cultured for 2 days, and the number of TRAP positive mononuclear cells present was determined.

Statistical Analysis

All experiments were performed in quadruplicate, and the mean±SEM for the number of OCLs formed was determined. The means of individual treatment groups were compared using Student's t-test, and the results were considered significantly different for p<0.01

### Results

Detection of ECF-L in Mature OCLs and Effects of ECF-L Conditioned Media on OCL Formation in Mouse Bone Marrow Cultures

Using PCR-selective subtraction hybridization, we detected approximately 200 bands that were overexpressed in mature OCLs compared with B/T precursor cells. Sequence analysis of the 68 bands that were most highly overexpressed in mature OCLs was performed as previously described Takahashi N, Yamana H, Yoshiki S, Roodman G D, Mundy G R, Jones S J, Boyde A, Suda T, 1988 Osteoclast-like cell formation and its regulation by osteotropic hormones in mouse bone marrow cultures. Endocrinology 122:1373-1382. We have reported the results of the first 50 bands Takahashi N, Yamana H, Yoshiki S, Roodman G D, Mundy G R, Jones S J, Boyde A, Suda T, 1988 Osteoclast-like cell formation and its regulation by osteotropic hormones in mouse bone marrow cultures. Endocrinology 122:1373-1382. DNA sequences of the other 18 bands were compared with the database in NCBI. Five bands were mouse complement component C3 of varying insert sizes, and one band was ECF-L.

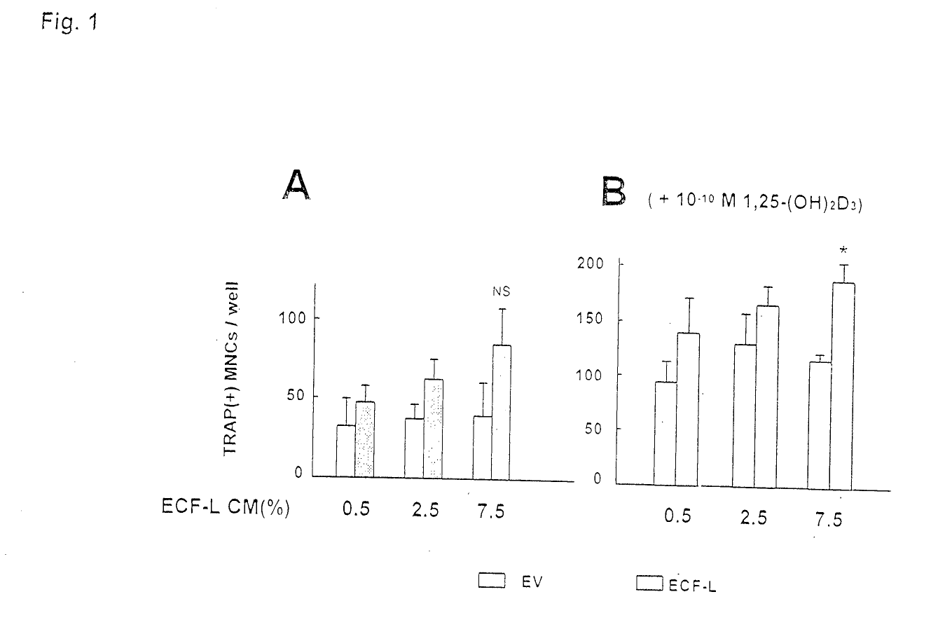

The full-length mouse ECF-L cDNA (1.3-kbp) was generated by PCR, its sequence confirmed by sequence analysis, and the PCR product then cloned into the mammalian expression vector pcDNA3. After transient transfection of mECF-L cDNA into 293 cells, the conditioned media were harvested and tested for their capacity to enhance TRAP(+) MNC formation in mouse bone marrow cultures in the presence or absence of low concentrations of 1,25-(OH)₂D₃ (10⁻¹⁰ M). With reference to FIG. 1, conditioned media from 293 cells transiently transfected with the cDNA for mECF-L induced OCL formation in murine bone marrow cultures. Varying concentrations of conditioned media were added to mouse bone marrow cultures in the absence (FIG. 1A) or in the presence (FIG. 1B) of 10⁻¹⁰ M of 1,25-(OH)₂D₃. At the end of the culture period, the cells were fixed and stained for TRAP activity, and the number of TRAP positive MNC was counted. ECF-L conditioned media significantly increased TRAP(+) MNC formation in a dose-dependent pattern in the presence of 10⁻¹⁰ M of 1,25-(OH)₂D₃. As compared with control conditioned media from 293 cells transfected with the empty vector. OCL-like MNC formation induced by ECF-L was enhanced by low concentrations of 1,25-(OH)₂D₃.

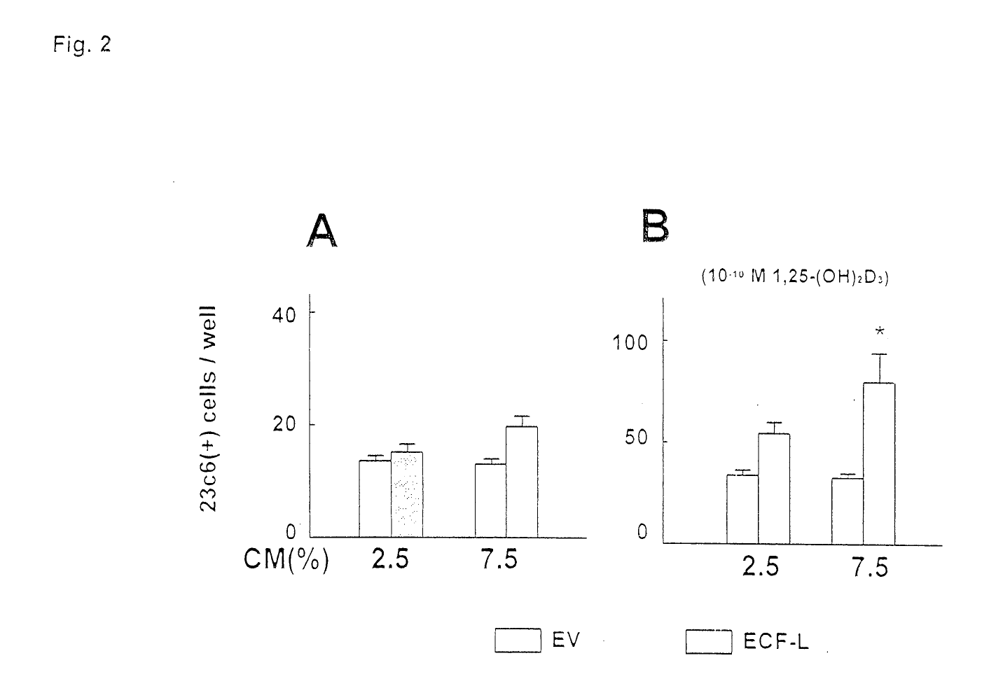

Referring to FIG. 2, conditioned media from 293 cells transiently transfected with the cDNA for human ECF-L induced OCL formation in human bone marrow cultures. Varying concentrations of conditioned media were added to human bone marrow cultures in the absence (FIG. 2A) or in the presence (FIG. 2B) of 10⁻¹⁰ M of 1,25-(OH)₂D₃. At the end of the culture period, the cells were fixed and stained with the 23c6 monoclonal antibody, and the number of 23c6 positive MNC was counted. Human ECF-L conditioned media significantly increased 23c6(+) MNC formation in a dose-dependent pattern in the presence of 10⁻¹⁰ M of a low concentration of 1,25-(OH)₂D₃ (10⁻¹⁰ M).

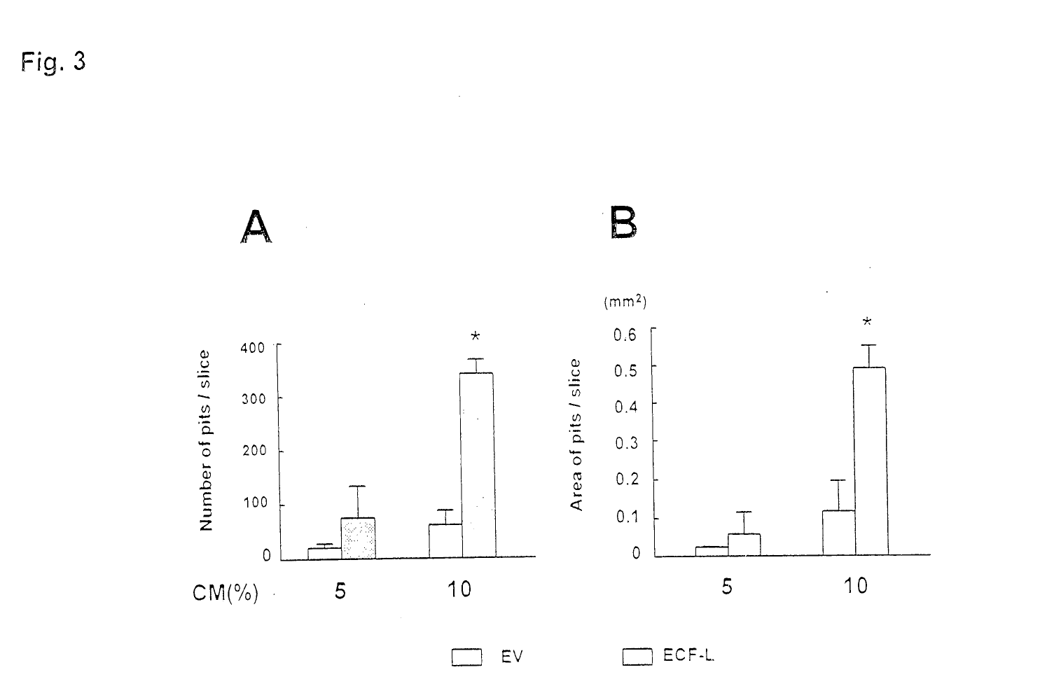

FIG. 3 relates to investigation of bone resorption capacity of OCL formed in mouse marrow cultures treated with conditioned media from 293 cells transiently transfected with the mECF-L cDNA. Mouse bone marrow cells were cultured on dentin slices in 48-well plates in the presence of 10⁻¹⁰ M of 1,25-(OH)₂D₃ and conditioned media from 293 cells transfected with ECF-L cDNA or empty vector (EV). After 9 days of culture, pit numbers (FIG. 3A) and pit area (FIG. 3B) per dentin slice were measured. OCL formed in cultures treated with ECF-L conditioned media in the presence of 1,25-(OH)₂D₃ (10⁻¹⁰ M) significantly increased resorption in a dose-dependent manner. As shown in FIG. 3, the number of pits and the resorption area per dentin slice were significantly increased by marrow cells treated with ECF-L conditioned media and 10⁻¹⁰ M 1,25-(OH)₂D₃ compared to those treated with the empty vector (EV) conditioned media and 1,25-(OH)₂D₃.

Time Course Effects of mECF-L Conditioned Media on OCL Formation in Mouse Bone Marrow Cultures

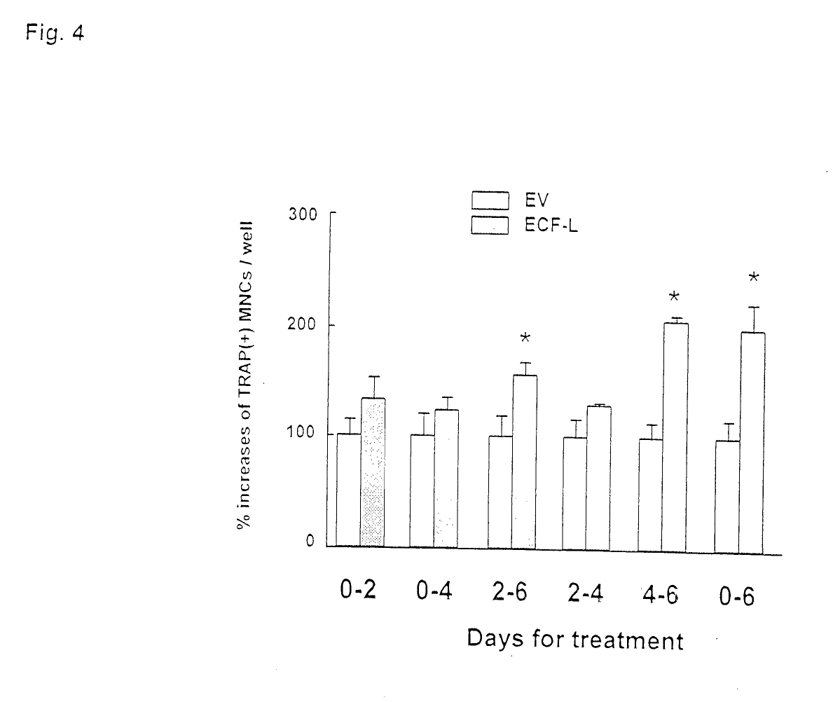

To determine whether ECF-L stimulated the proliferation or differentiation stage of OCL formation/activation, ECF-L conditioned media were added to more bone marrow cultures on days 0-2, 2-4, or for the entire 6 days of the culture in the presence of 10⁻¹⁰ M 1,25-(OH)₂D₃, and TRAP(+) MNC formation was determined. TRAP(+) MNC formation was significantly increased compared with the empty vector control media when ECF-L conditioned media were present for the later stage (days 4-6) of the culture or for the entire culture period. ECF-L conditioned media did not increase MNC formation if present only during the early stages of the culture (FIG. 4).

Effects of Antisense S-oligonucleotide to mECF-L on OCL Formation in Mouse Bone Marrow Cultures

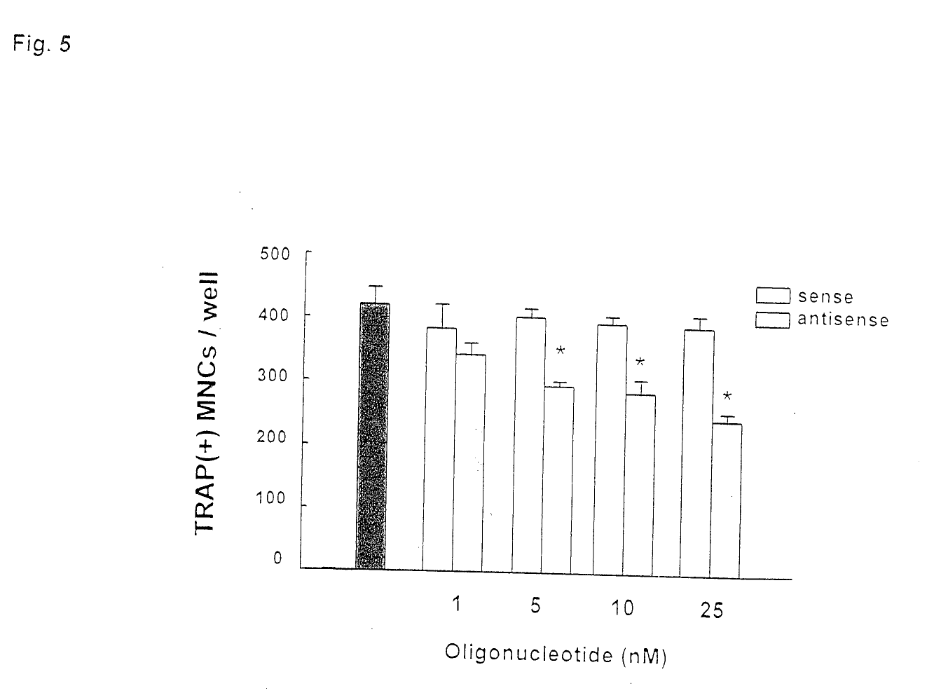

To determine if endogenous ECF-L was playing a role in OCL formation/activation, we tested the effects of various concentrations of sense of an antisense S-oligonucleotide to ECF-L on MNC formation in murine cultures treated with 1,25-(OH)₂D₃ (10^{31 9} M). Antisense S-oligonucleotide to ECF-L (5-25 nM) significantly inhibited OCL formation by about 40% in murine bone marrow cultures stimulated with 10⁻⁹ M 1,25-(OH)₂D₃ compared with the control cultures treated with sense S-oligonucleotide (FIG. 5). High concentrations of S-oligonucleotide (more than 50 nM) were toxic to murine bone marrow cultures.

In situ Hybridization

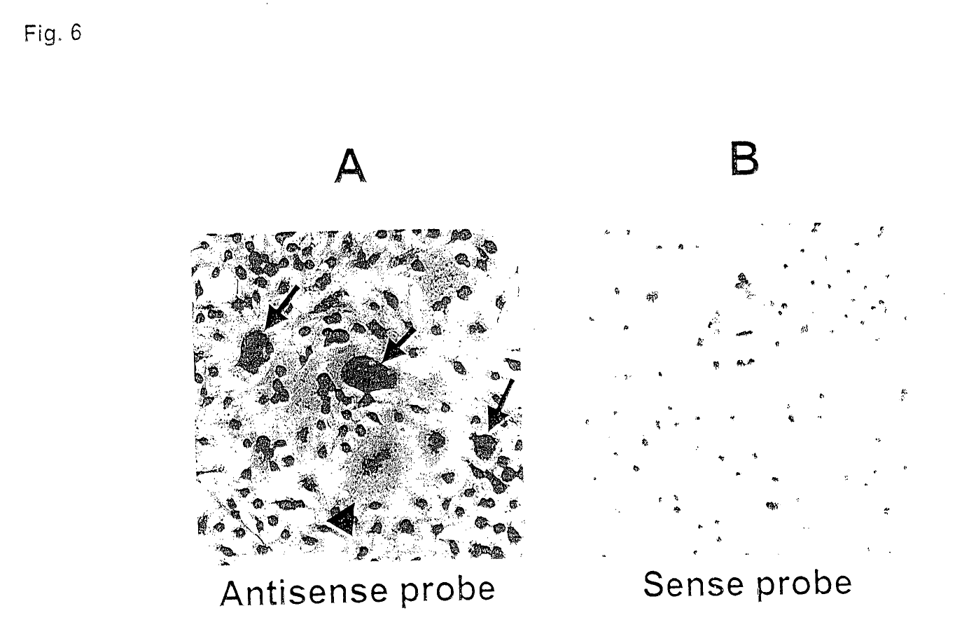

To identify the cells that express ECF-L, we performed in situ hybridization on mouse bone marrow cultures with a DIG-labeled cRNA probe to ECF-L. As shown in FIG. 6A (antisense probes), the expression of ECF-L mRNA was detected in monocytes and multinucleated cells (MNCs), but not in fibroblasts. In contrast, no signal was detected in control cultures hybridized with sense RNA probes (arrow) (FIG. 6B). Interestingly, MNC that contained less than 5 nucleus strongly expressed ECF-L, while MNC that contained more than 10 nucleus did not express ECF-L (arrow head).

Western Blot Analysis

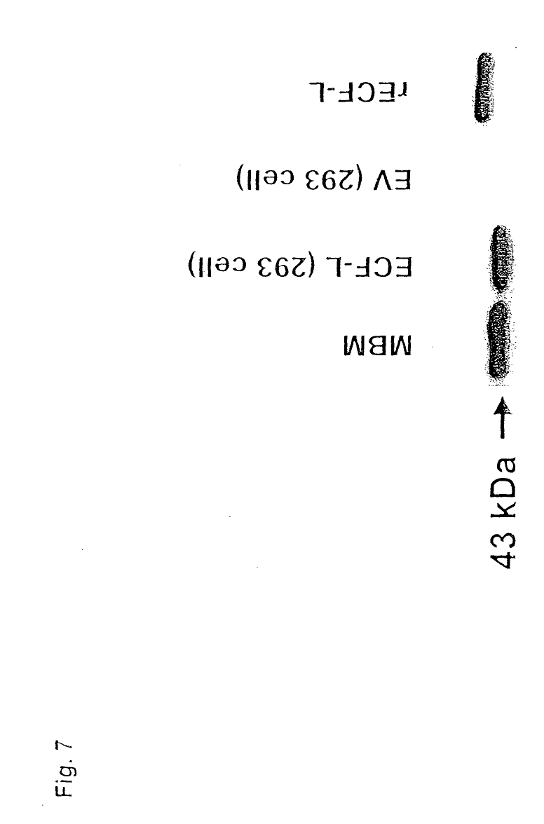

To determine if murine bone marrow cells secrete ECF-L into their conditioned media, we performed Western blot analysis with conditioned media from mouse bone marrow cultures treated with 1,25(OH)₂D₃ or from 293 cells transfected with the ECF-L cDNA or empty vector, using polyclonal rmECF-L antisera raised in rabbits. As shown in FIG. 7, a 43 kDa band was detected in conditioned media from mouse bone marrow treated with 10⁻⁹ M 1,25-(OH)₂D₃ and 293 cells transiently transfected with ECF-L cDNA, but not in conditioned media from 293 cells transfected with the empty vector.

Effect of ECF-L Antisera in TRAP(+) MNC Formation in Mouse Bone Marrow Cultures

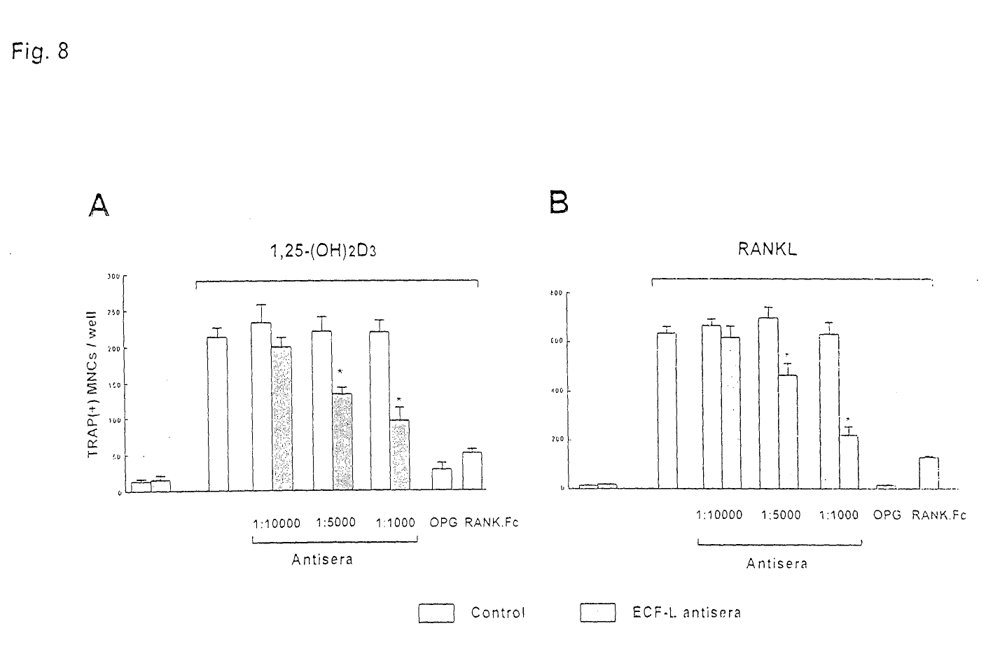

To confirm the role of ECF-L in OCL formation, the effects of ECF-L polyclonal antisera were tested in mouse bone marrow cultures stimulated with 10^{31 9} M 1,25-(OH)₂D₃ or 20 ng/ml RANKL. OPG and RANK-Fc were added to the cultures at concentrations of 50 ng/ml. After seven days, the cells were fixed and stained for TRAP. OCL formation induced by 1,25-(OH)₂D₃ (FIG. 8A) and RANKL (FIG. 8B) was dose-dependently inhibited about 60% by anti-ECF-L antibody at 1:10,000-1:1,000 dilution.

Effects of rECF-L-Fc Fusion Protein on OCL Formation/activation in Mouse Bone Marrow Cultures

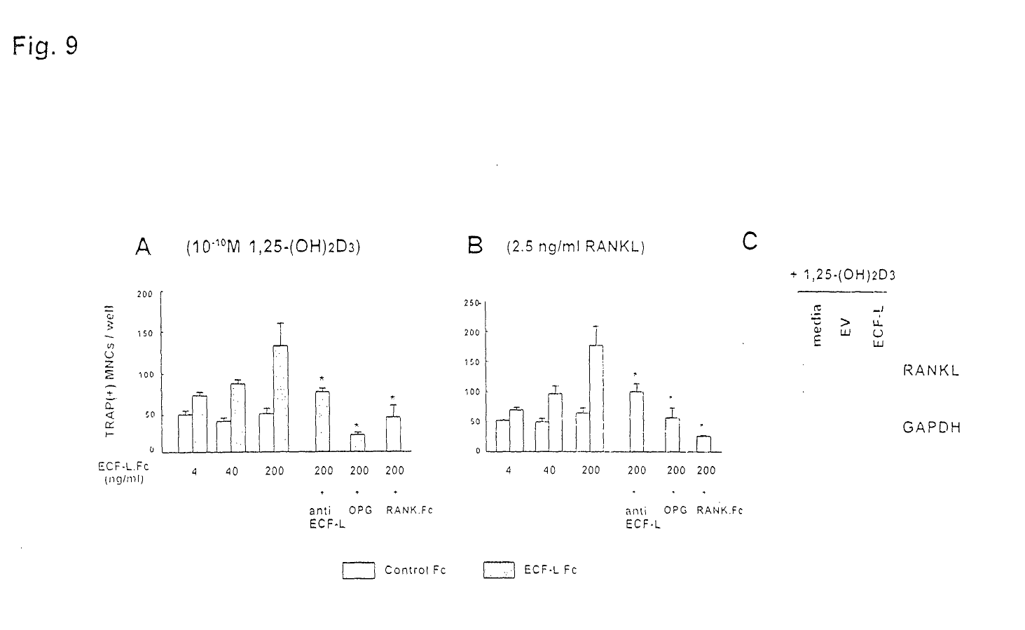

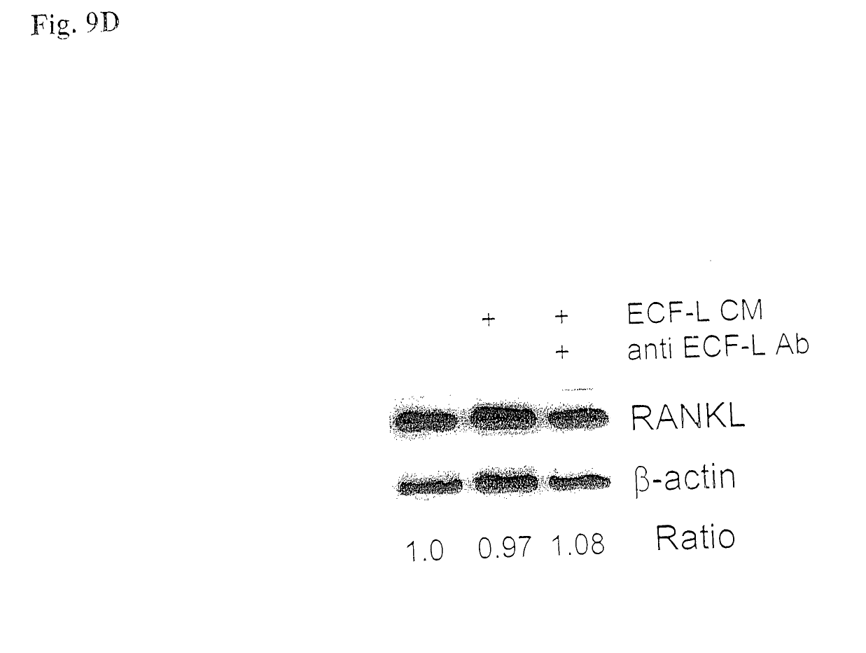

We tested the effects of rECF-L-Fc fusion protein on OCL formation in mouse bone marrow cultures. rECF-L-Fc fusion protein induced TRAP(+) MNC formation in the presence of 10⁻¹⁰ M 1,25-(OH)₂D₃ (FIG. 9A) or 2.5 ng/ml RANKL (FIG. 9B) in the dose-dependent manner (4-200 ng/ml) compared to the control Fc protein.

In order to determine if OCL formation induced by ECF-L occurred via the RANK-RANKL pathway, the effects of OPG and RANK-Fc on OCL formation stimulated with ECF-L were examined. Varying concentrations of recombinant ECF-L-Fc fusion protein were added to mouse bone marrow cultures in the absence or in the presence of 10⁻¹⁰ M of 1,25-(OH)₂ D₃ (A) or 2.5 ng/ml RANKL (B). Anti ECF-L antisera were used at 1:1000 dilution, and OPG or RANK-Fc were added to the cultures at a concentration of 50 ng/ml. After 7 days, the cells were fixed and stained for TRAP. Purified ECF-L-Fc fusion protein enhanced TRAP(+) MNC formation in a dose-dependent pattern, and this effect was blocked by ECF-L antisera, OPG and RANK-Fc. OPG and RANK-Fc significantly inhibited OCL formation induced by ECF-L-Fc in the presence of 10⁻¹⁰ M 1,25-(OH)₂D₃ (9A) or 2.5 ng/ml RANKL (FIG. 9B). Mouse bone marrow cultures were treated with ECF-L conditioned media in the presence of 10⁻¹⁰ M 1,25-(OH)₂ D₃ for 2 days. Total RNA was isolated as described in Methods and RT-PCR analysis for murine RANKL was performed. RANKL mRNA levels were not increased by ECF-L. GAPDH was used as an internal control for the RT-PCR. However, ECF-L did not enhance RANKL mRNA expression induced by 10⁻¹⁰M 1,25-(OH)₂D₃ compared to control cultures treated with empty vector conditioned media (FIG. 9C). Effects of ECF-L conditioned media on RANKL expression. Lysates from mouse bone marrow cells treated with ECF-L conditioned media and 10⁻¹⁰ M 1,25(OH)₂D₃ with or without ECF-L antibody were analyzed by Western blot analysis as described in methods. ECF-L did not enhance RANKL expression. The ratios of the RANKL band to the β-actin band were 1.0 (non-treated), 0.97 (10% ECF-L conditioned media), and 1.08 (10% ECF-L conditioned media and 1:1,000 ECF-L antibody) respectively. Furthermore, Western blot analysis showed that the expression levels of RANKL in the presence of 10⁻¹⁰M 1,25-(OH)₂D₃ were not significantly enhanced by ECF-L CM or decreased by ECF-L antibody compared to the β-actin internal control (FIG. 9D).

Chemotaxis Assays

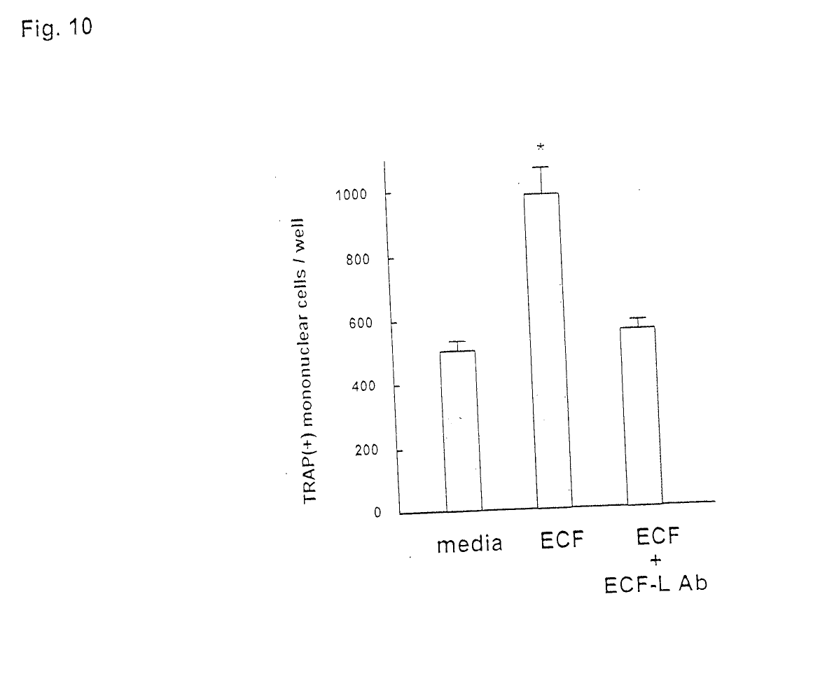

To test the chemotactic effects of ECF-L on OCL precursors, we performed chemotactic assays. Chemotactic activity of recombinant ECF-L cells were treated with ECF-L in the presence of 10⁻¹⁰ M of 1,25-(OH)2 D₃. ECF-L (400 ng/mL) was chemotactic for TRAP(+)OCL precursors compared to control cultures, and the chemoattractant activity was completely blocked by adding ECF-L antisera (1:1000). As shown in FIG. 10, recombinant ECF-L showed chemotactic activity for OCL precursors compared to control cultures. Neutralizing mECF-L with the ECF-L antisera blocked the chemoattract effects of ECF-L on OCL precursors.

### Discussion

PCR-selective subtraction is a powerful technique for identifying genes that are overexpressed in mature OCLs compared with OCL precursors. Using this technique, we detected mouse ECF-L. We confirmed by in situ hybridization that ECF-L mRNA was highly expressed in OCLs that had less than five nuclei and in marrow mononuclear cells.

ECF-L was first identified as a novel eosinophil chemotactic cytokine by Owhashi et al Owhashi M, Arita H, Hayai N, 2000 Identification of a novel eosinophil chemotactic cytokine (ECF-L) as a chitinase family protein. J Biol Chem 275:1279-1286. ECF-L possesses a CXC sequence near the NH2 terminus of the mature molecule, which is a typical motif shared with many chemokine family proteins. Sequence alignments revealed that ECF-L differs from other known eosinophil chemotactic cytokines such as interleukine-5 Kinashi T, Harada N, Severinson E, Tanabe T, Sideras P, Konishi M, Azuma C, Tominaga A, Bergstedt-Lindqvist S, Takahashi M, Matsuda F, Yaoita Y, Takatsu K, Honjo T, 1986 Cloning of complementary DNA encoding T-cell replacing factor and identity with B-cell growth factor II. Nature 324:70-73., RANTES Schall T J, Simpson N J, Mak J Y, 1992 Molecular cloning and expression of the murine RANTES cytokine: Structural and functional conservation between mouse and man. Eur J Immol 22:1477-1481., eotaxin Rothenberg M E, Luster A D, Leder P, 1995 Murin eotaxin: An eosinophil chemoattractant inducible in endothelial cells and interleukin 4-induced tumor suppression. Proc Natl Acad Sci USA 92:8960-8964., or ecalectin Matsumoto R, Matsumoto H, Seki M, Hata M, Asano Y, Kanegasaki S, Stevens R L, Hirashima M, 1998 Human ecalectin, a variant of human galectin-9, is a novel eosinophil chemoattractant produced by T lymphocytes. J Biol Chem 273:16976-16984. Comparisons of the deduced amino acid sequence with those contained in several databases revealed that ECF-L had a high homology with the chitinase family of 18 glycosyl hydrolases and vertebrate chitinase family proteins that do not demonstrate chitinase activity Owhashi M, Arita H, Hayai N, 2000 Identification of a novel eosinophil chemotactic cytokine (ECF-L) as a chitinase family protein. J Biol Chem 275:1279-1286. Although proteins of the chitinase family are detected in mammals, no chitinolytic activity has been detected Elias J A, Tang W, Horowitz M C, 1995 Cytokine and hormonal stimulation of human osteosarcoma interleukin-11 production. Endocrinology 136:489-498, and the actual physiological roles of the mammalian chitinases family proteins remain to be clarified.

Our study demonstrated that ECF-L enhanced OCL formation in the presence of low concentrations of osteotropic factors such as 1,25-(OH)₂D₃ and RANKL in mouse bone marrow cultures. Similarly, IL-6 enhances only proliferation of OCL precursors in murine culture systems and requires other osteoclastogenetic factors such as 1,25(OH)₂D₃ or PTHrP to induce murine OCL formation Kukita T, Nakao J, Hamada F, Kukita A, Inai T, Kurisu K, Noriyama H, 1992 Recombinant LD78 protein, a member of small cytokine family, enhances osteoclast differentiation in rat bone marrow culture system. Bone and Miner 19:215-223. Kukita and coworkers Baggiolini M, Dewald B, Moser B., 1997 Human chemokines: an update. Annu Rev Immunol 15:675-705 also showed MIP-1□ induced formation of OCLs in rat bone marrow cultures was dependent on 1,25-(OH)₂D₃. These OCL stimulatory effects may be mediated by the production of other osteoclastogenic factors such as RANKL.

Time-course studies suggested that ECF-L acts at the later stages of OCL formation such as the cell fusion stage rather than inducing proliferation of OCL precursors. Blocking ECF-L expression in marrow cultures inhibits OCL formation, suggesting an important role for ECF-L in the later stages of osteoclastogenesis. ECF-L may act as a chemoattractant for OCL precursors, as demonstrated by chemotactic assays with ECF-L.

ECT-L appears to play an important role in RANK-L mediated osteoclastogenesis. ECF-L enhanced RANK-L induced OCL formation, and ECF-L antisera blocked OCL formation induced by RANKL. In addition, OPG or RANK-Fc inhibited ECF-L enhanced OCL formation. However, the effects of ECF-L on OCL formation were not due to increased expression of RANKL or RANK, since ECF-L did not increase RANKL levels in mouse bone marrow cultures treated with 1,25-(OH)₂D₃, using either RT-PCR or Western blot analysis. Furthermore, ECF-L antisera did not affect the expression of RANK (data not shown). These data suggest that ECF-L requires RANKL to induce OCL formation, but is itself an important cofactor involved in RANKL induced OCL formation, possibly though its chemotactic effects on OCL precursors.

Chemokines activate cells by binding to specific cell-surface receptors that belong to a superfamily of serpentine G-protein-coupled receptors, and the receptor binding profiles of various chemokines have been reviewed Baggiolini M, Dewald B, Moser B., 1997 Human chemokines: an update. Annu Rev Immunol 15:675-705; Kunkel S L, 1999 Through the looking glass: the diverse in vivo activities of chemokines. J Clin Invest 104:1333-1334. Votta and coworkers Votta B J, White J R, Dodds R A, James I E, Conner J R, Lee-Rykaczweski E, Eichman C F, Kumar S, Lark M W, Gowen M, 2000 CK□-8(CCL23), a novel CC chemokine, is chemotactic for human osteoclast precursors and is expressed in bone tissue. J Cell Phys 183:196-207 showed that a novel chemokine, CK-□8 (recently designated as CCL23; and previously described as myeloid progenitor inhibitory factor-1, MPIF-1), was a chemotactic factor for human OCL precursors. CCR1 appears to be the primary receptor on monocytes and eosinophils through which CK-□8 signaling is transduced Forssmann U, Delgado M B, Uguccioni M, Loestcher P, Garrotta G, Bagiolini M, 1997 CK□-8, a novel cc chemokine that predominately acts on monocytes. FEBS Lett 408:211-216. ECF-L has been reported to have a specificity similar to RANTES as a chemoattractant for eosinophils, T lymphocytes, and bone marrow cells, and this result indicates that the receptor(s) for ECF-L is related to that for RANTES Owhashi M, Arita H, Hayai N, 2000 Identification of a novel eosinophil chemotactic cytokine (ECF-L) as a chitinase family protein. J Biol Chem 275:1279-1286. However, RANTES binds to multiple chemokine receptors (CCR1, CCR3, CCR4, and CCR5). Thus, the identification of receptor mediating the effects of ECF-L remains to be clarified.

In summary, ECF-L is a recently identified chemokine that is a chemoattractant for OCL precursors. ECF-L is highly expressed in OCL and mononuclear OCL precursors and enhances OCL formation induced by RANKL. However, ECF-L acts independently of RANKL, but appears to play an important role in RANK induced OCL formation.

Other embodiments of the invention include the following genbank accession sequence numbers.

YKL-39: LOCUS NM_{—}004000 1418 bp mRNA linear PRI 03-APR-2003 DEFINITION Homo sapiens chitinase 3-like 2 (CHI3L2), mRNA. ACCESSION NM_{—}004000 VERSION NM_{—}004000.1 GI:11993934

TSA-19029(S): LOCUS AB025009 1188 bp mRNA linear PRI 25-NOV-1999 DEFINITION Homo sapiens

TSA1902-S mRNA for novel member of chitinase family, complete cds. ACCESSION AB025009 VERSION AB025009.1 GI:6467178 KEYWORDS novel member of chitinase family; TSA1902-S. SOURCE Homo sapiens (human)

TSA-1902(L): LOCUS AB025008 1354 bp mRNA linear PRI 25-NOV-1999 DEFINITION Homo sapiens TSA1902-L mRNA for novel member of chitinase family, complete cds. ACCESSION AB025008 VERSION AB025008.1 GI:6467176 KEYWORDS novel member of chitinase family; TSA1902-L. SOURCE Homo sapiens (human)

Note: TSA1092(L)=the EST that Roodman identified as being the putative human homolog of mouse ECF-L. TSA1902(L) is partial—its sequence is contained within acidic mammalian chitinase

### Acknowledgement

These studies were supported by R01-AR41336 from NIAMS and funds from the MMRF.

Whereas particular embodiments of the invention have been described herein for purposes of illustration, it will be evident to those skilled in the art that numerous variations of the details may be made without departing from the invention as defined in the appended claims.

1. A method of resisting osteoclast formation comprising inhibiting eosinophil chemotactic factor-L expression or activity.

2. The method of claim 1, including
effecting said inhibiting by means of an anti-ECF-L antibody.

3. The method of claim 1, including
effecting said inhibiting by antisense S-oligonucleotide to ECF-L.

4. The method of claim 1, including
effecting said inhibition by mECF-L polyclonal antisera.

5. The method of claim 1, including
effecting said inhibiting by rabbit preimmune antisera.

6. The method of claim 1, including
effecting said inhibiting by OPG.

7. The method of claim 1, including
effecting said inhibiting by RANK-Fc.

8. The method of claim 1, including
employing said method in human cells.

9. The method of claim 8, including
employing said method in vivo.

10. A method of resisting osteoclast formation comprising
inhibiting RANKL expression or activity.

11. The method of claim 10, including
effecting said inhibiting by means of an anti-ECF-L antibody.

12. The method of claim 11, including
effecting said inhibiting by means of polyclonal antisera.

13. The method of claim 9, including
effecting said inhibiting on human cells.

14. The method of claim 13, including
employing said method in vivo.

15. A method of resisting osteoclast formation comprising
inhibiting mECF-L activity in the presence of RANKL.

16. The method of claim 15, including
effecting said inhibiting by use of anti-RANKL polyclonal antibody.

17. The method of claim 16, including
effecting said inhibiting by means of OPG.

18. The method of claim 16, including
effecting said inhibiting by means of RANK-Fc.

19. The method of claim 16, including
effecting said inhibiting on human cells.

20. The method of claim 19, including
employing said method in vivo.

21. The method of claim 2 or 11, wherein the antibody is a monoclonal antibody or active fragment thereof.

22. The method of claim 21, wherein the antibody or antibody fragment is human.

23. The method of claim 22, wherein the antibody or antibody fragment is humanized.

24. An isolated anti-ECF-L antibody or fragment thereof capable of inhibiting or neutralizing ECF-L activity.

25. The antibody or fragment thereof of claim 24, capable of inhibiting ECF-L induced osteoclast formation.

26. The antibody of claim 24 or 25, wherein said antibody is monoclonal or an active fragment thereof.

27. The antibody or fragment of claim 26 which is human.

28. The antibody or fragment of claim 26 which is humanized.


SEQUENCE LISTING (12 sequences)

SEQ ID NO: 1 - DNA, 20 bp, Artificial Sequence
  Description of Artificial Sequence Synthetic primer
acaccatggccaagctcatt

SEQ ID NO: 2 - DNA, 20 bp, Artificial Sequence
  Description of Artificial Sequence Synthetic primer
tgcagaatgcgctgtggaaa

SEQ ID NO: 3 - DNA, 30 bp, Artificial Sequence
  Description of Artificial Sequence Synthetic oligonucleotide
aagaatgagcttggccatggtgtcttcacg

SEQ ID NO: 4 - DNA, 30 bp, Artificial Sequence
  Description of Artificial Sequence Synthetic oligonucleotide
cgtgaagacaccatggccaagctcattctt

SEQ ID NO: 5 - DNA, 31 bp, Artificial Sequence
  Description of Artificial Sequence Synthetic primer
cgaggatccgatggccaagctcattcttgtc

SEQ ID NO: 6 - DNA, 30 bp, Artificial Sequence
  Description of Artificial Sequence Synthetic primer
cgaggatcctcaataagggcccttgcaact

SEQ ID NO: 7 - protein, 6 aa, Artificial Sequence
  Description of Artificial Sequence Synthetic peptide
HHHHHH

SEQ ID NO: 8 - DNA, 30 bp, Artificial Sequence
  Description of Artificial Sequence Synthetic primer
atcgtaatccataagggcccttgcaacttg

SEQ ID NO: 9 - DNA, 20 bp, Artificial Sequence
  Description of Artificial Sequence Synthetic primer
gaaggtactcgtagctaagg

SEQ ID NO: 10 - DNA, 20 bp, Artificial Sequence
  Description of Artificial Sequence Synthetic primer
ggctatgtcagctcctaaag

SEQ ID NO: 11 - DNA, 20 bp, Artificial Sequence
  Description of Artificial Sequence Synthetic primer
accacagtccatgccatcac

SEQ ID NO: 12 - DNA, 20 bp, Artificial Sequence
  Description of Artificial Sequence Synthetic primer
tccaccaccctgttgctgta